(19) United States
(12) Patent Application Publication
(10) Pub. No.: US 2008/0229447 A1
(43) Pub. Date: Sep. 18, 2008

(54) Transformation of immature soybean seeds through organogenesis

(75) Inventors: YuehJiang Hwang, Chapel Hill, NC (US); John Dawson, Greensboro, NC (US); Marina Sigareva, Chapel Hill, NC (US); Qiudeng Que, Cary, NC (US)

Correspondence Address: JENKINS, WILSON, TAYLOR & HUNT, P. A., Suite 1200 UNIVERSITY TOWER, 3100 TOWER BLVD.,, DURHAM, NC, 27707, US

(73) Assignee: Syngenta Participations AG, Basel (CH)

(21) Appl. No.: 11/716,975

(22) Filed: Mar. 12, 2007

(51) Int. Cl. A01H 5/00; C12N 15/87; C12N 5/04

(52) U.S. Cl. 800/279; 800/278; 800/294; 800/312

(57) ABSTRACT

The present disclosure provides methods for the transformation of soybean cells or tissue and regeneration of the soybean cells or tissue into transformed plants. The disclosed methods utilize an explant prepared from an immature soybean seedling which can be induced directly to form shoots that give rise to transgenic plants via organogenesis. The disclosed methods do not require germination and are rapid and efficient.

### TECHNICAL FIELD

The presently disclosed subject matter relates generally to methods for plant transformation and, more particularly, to methods for transforming soybean cells or tissues using organogenesis. The presently disclosed subject matter also relates to methods for regenerating transgenic soybean plants from transformed soybean cells or tissues. The presently disclosed subject matter further relates to transgenic soybean plants and seeds obtained by such methods.

TABLE OF ABBREVIATIONS

| | |
|---|---|
| A | absorbance |
| BAP | 6-Benzylaminopurine |
| cm | centimeter |
| DNA | deoxyribonucleic acid |
| g | gram |
| GFP | green fluorescence protein |
| GH | greenhouse |
| HPPD | 4-hydroxyphenylpyruvate dioxygenase |
| IAA | indole-3-acetic acid |
| IBA | 3-indolebutyric acid |
| ID | identification |
| L | liter |
| M | molar |
| m² | meters squared |
| MES | 2-(N-morpholino)ethanesulfonic acid |
| mg | milligram |
| min | minute |
| ml | milliliter |
| MS | Murshige & Skoog |
| NAA | 1-naphthaleneacetic acid |
| No. | number |
| sec | second |
| μE | microeinsteins |
| μM | micromolar |
| ° C. | degrees Celsius |
| % | percent |
| > | greater than |
| < | less than |
| ≧ | greater than or equal to |
| ≦ | less than or equal to |

### BACKGROUND

Cultivated soybean (Glycine max) is a major food and feed crop, with a substantial commercial value throughout the world. Over 50 million hectares worldwide are used to produce an annual crop of soybeans in excess of 100 metric tons with an estimated value exceeding 20 billion dollars. Unfortunately, only a few plant introductions have given rise to the major cultivars grown in the United States and, as a consequence, the narrow germplasm base has limited soybean breeding potential. The limited genetic base in domestic soybean varieties has limited the power of traditional breeding methods to develop varieties with improved or value-added traits. The development of scientific methods useful in improving the quantity and quality of this crop is therefore of significant commercial interest.

Modern biotechnological research and development have provided useful techniques for the improvement of agricultural products by plant genetic engineering. Plant genetic engineering involves the transfer of a desired gene or genes into the inheritable germline of crop plants such that those genes can be bred into or among the elite varieties used in modern agriculture. Gene transfer techniques allow the development of new classes of elite crop varieties with improved disease resistance, herbicide tolerance, and increased nutritional value. Various methods have been developed for transferring genes into plant tissues, including high velocity microprojection, microinjection, electroporation, direct DNA uptake, and Agrobacterium-mediated gene transformation.

Transformation systems employing the bacterium Agrobacterium tumefaciens have conventionally been used for the genetic transformation of soybean plants. In addition, high velocity microprojectile bombardment has also been used as an alternative method for the genetic transformation of soybean plants.

Although advances have been made in the field of plant transformation, a need continues to exist for improved methods to facilitate the ease, speed, and efficiency of such methods for the transformation of soybean plants.

### SUMMARY

The presently disclosed subject matter provides an organogenic method for transforming soybean cells or tissues. In some embodiments, the methods comprise preparing an explant from an immature soybean seed, contacting the explant with a genetic construct, and culturing the explant in the presence of a selection agent.

In some embodiments, preparing the explant comprises one or more of the following: (a) isolating seeds from seed pods and sterilizing the seeds; (b) preparing transformation target explant tissues by one of the following: (i) removing from the seed its seed coat, one cotyledon, and two primary leaves; (ii) removing the hypocotyl from the seed, removing the seed coat, and removing one cotyledon and two primary leaves; (iii) removing the hypocotyl from the seed, removing all or part of the seed coat, bisecting the immature soybean seed longitudinally through an embryo axis, whereby both halves of the explant include one cotyledon and part of the immature embryo axis; (iv) removing the seed coat; and (v) removing the seed coat, and removing at least part of two cotyledons and two primary leaves.

In some embodiments, the one or more methods of preparing the explants further comprise wounding a plumule, cotetylenary node region, hypocotyl, or combination thereof in the explant.

In some embodiments, the immature soybean seed is selected from developmental stage late R5, R6, or R7.

In some embodiments, the genetic construct comprises a selectable marker gene.

In some embodiments, the genetic construct comprises a gene of interest and/or a selectable marker gene.

In some embodiments, the selectable marker gene confers antibiotic or herbicide resistance to the explant.

In some embodiments, the antibiotic is selected from the group consisting of: cefotaxime, timetin, vancomycin, carbenicillin, gentamicin, kanamycin, streptomycin, azithromycin, erythromycin, penicillin G, penicillin V, oxacillin, cloxacillin, dicloxacillin, ampicillin, amoxicillin, ticarcillin, ciprofloxacin, doxycycline, minocycline, tetracycline, vancomycin, and combinations thereof.

In some embodiments, the herbicide is selected from the group consisting of: glyphosate, sulfonylurea, imidazolinone, glufosinate, bialophos, phenoxy proprionic acid, cycloshexone, triazine, benzonitrile, HPPD inhibitors, and combinations thereof.

In some embodiments, the genetic construct comprises a DNA molecule delivered by an Agrobacterium cell.

In some embodiments, the Agrobacterium is Agrobacterium tumefaciens.

In some embodiments, the explant is contacted with the Agrobacterium cells, preferably for up to about 24 hours.

In some embodiments, the contacting comprises delivering the genetic construct to the explant using a ballistic device.

In some embodiments, the presently disclosed methods comprise culturing the explant on a culture medium comprising a plant hormone.

In some embodiments, the explants are further co-cultured with Agrobacterium cells for up to about 7 days.

In some embodiments, the presently disclosed methods comprise culturing the transformed shoot on a shooting medium comprising a plant hormone.

In some embodiments, the presently disclosed methods comprise regenerating a shoot into a genetically transformed soybean plant.

In some embodiments, the presently disclosed subject matter comprises transgenic soybean cells or tissues prepared according to the disclosed methods.

In some embodiments, the presently disclosed subject matter comprises soybean plants regenerated from the transgenic soybean cells or tissues of the disclosed methods.

In some embodiments, the presently disclosed subject matter comprises transgenic seeds produced by the disclosed soybean plants.

Accordingly, it is an object of the presently disclosed subject matter to provide for the transformation of immature soybean seeds through organogenesis.

An object of the presently disclosed subject matter having been stated hereinabove, and which is achieved in whole or in part by the presently disclosed subject matter, other objects will become evident as the description proceeds.

### DETAILED DESCRIPTION

### I. GENERAL CONSIDERATIONS

Numerous methods for plant transformation have been developed, including biological and physical plant transformation protocols. See, for example, Miki et al., “Procedures for Introducing Foreign DNA into Plants” in Methods in Plant Molecular Biology and Biotechnology, Glick, B. R. and Thompson, J. E. Eds. (CRC Press, Inc., Boca Raton, 1993) pp. 67-88. In addition, expression vectors and in vitro culture methods for plant cell or tissue transformation and regeneration of plants are available. See, for example, Gruber et al., “Vectors for Plant Transformation” in Methods in Plant Molecular Biology and Biotechnology, Glick, B. R. and Thompson, J. E. Eds. (CRC Press, Inc., Boca Raton, 1993) pp. 89-119.

However, to date, no comprehensive mechanism for the organogenic transformation of soybean has been presented. In particular, an approach where immature soybean seed explants are directly transformed via organogenesis pathway and the omission of the seed germination phase is possible have not been disclosed in the art. As disclosed in detail herein, the present subject matter provides in some embodiments methods for the direct organogenic transformation of soybean, Glycine max. The disclosed methods are based on gene delivery into cells of explants prepared from immature soybean seeds. The transformed cells are then induced to form shoots that, at a high frequency, can be cultivated into whole sexually mature and fertile transgenic soybean plants. The disclosed methods do not involve a seed germination or pre-culture phase, and hence the time period of the entire process of preparing transformation targets can be remarkably concise.

Accordingly, provided herein are methods whereby transgenic plants are generated through an organogenesis approach using immature seeds directly isolated from pods as a source for explants for genetic transformation. Currently in the art, organogenesis is mediated by the use of mature and dry seeds as starting materials and can involve an imbibition or germination process to obtain seedlings for preparing target tissue for transformation. However, in the presently disclosed methods, immature or undessicated mature seeds can be used as starting material for preparing target explants, thereby eliminating the imbibition or pre-culture requirement.

Currently in the art, young cotyledons from immature embryos or seeds are only used for transformation through an embryogenesis approach. See, for example, U.S. Pat. Nos. 6,858,777 and 5,569,834. However, the embryogenesis approach can typically require a delayed time period from inoculation to rooted transgenic plants, of up to 10 months. In comparison, the methods of presently disclosed subject matter allows for a notably shorter timeline through the transformation of cells from immature seeds via organogenesis.

### II. DEFINITIONS

Unless otherwise defined, all technical and scientific terms used herein have the same meaning as commonly understood by one of ordinary skill in the art to which the presently disclosed subject matter pertains. For clarity of the present specification, certain definitions are presented herein below.

Following long-standing patent law convention, the terms “a” and “an” mean “one or more” when used in the subject application, including the claims.

As used herein, the term “about”, when referring to a value or to an amount of mass, weight, time, volume, concentration or percentage is meant to encompass variations of ±20% or ±10%, in another example 5%, in another example ±1%, and in still another example ±0.1% from the specified amount, as such variations are appropriate to practice the presently disclosed subject matter. Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “chimeric polypeptide” refers to a polypeptide that comprises domains or other features that are derived from different polypeptides or are in a position relative to each other that is not naturally occurring. Accordingly, the term “chimeric construct” refers to a recombinant nucleic acid molecule in which a promoter or regulatory nucleic acid sequence is operatively linked to, or associated with, a nucleic acid sequence that codes for an mRNA or which is expressed as a polypeptide, such that the regulatory nucleic acid sequence is able to regulate transcription or expression of the associated nucleic acid sequence. The regulatory nucleic acid sequence of the chimeric construct is not normally operatively linked to the associated nucleic acid sequence as found in nature.

As used herein, the term “cotyledon” refers to the first, first pair, or first whorl of leaf-like structures on a plant embryo that function primarily to make food compounds in the seed available to the developing totipotent plant tissue.

The term “cotyledonary node” or “cot-node” as used herein refers to the region in the embryo axis where cotyledons attach to, usually including 0.2 cm above and below the cotyledon attachment point in the immature seeds.

As used herein, the term “DNA segment” refers to a DNA molecule that has been isolated free of total genomic DNA of a particular species. Included within the term “DNA segment” are DNA segments, smaller fragments of such segments, and recombinant vectors, including but not limited to plasmids, cosmids, phages, viruses, and the like.

As used herein, the phrase “enhancer-promoter” refers to a composite unit that contains both enhancer and promoter elements. An enhancer-promoter is operatively linked to a coding sequence that encodes at least one gene product.

The term “epicotyl” as used herein refers to the portion of a plant located between cotyledonary node and primary leaf node.

As used herein, the term “embryo axis” refers to the embryo organs or parts including the plumule, epicotyl, cotyledonary node, hypocotyl, and radicle. In some embodiments, the term “embryo axis” refers to the longitudinal central line around which the organs or parts of the embryo are arranged.

As used herein, the term “expression cassette” refers to a nucleic acid molecule capable of directing expression of a particular nucleotide sequence in an appropriate host cell, comprising a promoter operatively linked to the nucleotide sequence of interest which is operatively linked to termination signals. It also can comprise sequences required for proper translation of the nucleotide sequence. The coding region can encode a polypeptide of interest and can also encode a functional RNA of interest, including but not limited to, antisense RNA or a non-translated RNA, in the sense or antisense direction. The expression cassette comprising the nucleotide sequence of interest can be chimeric, meaning that at least one of its components is heterologous with respect to at least one of its other components. The expression cassette can also be one that is naturally occurring but has been obtained in a recombinant form useful for heterologous expression. In some embodiments, however, the expression cassette is heterologous with respect to the host; i.e., the particular DNA sequence of the expression cassette does not occur naturally in the host cell and was introduced into the host cell or an ancestor of the host cell by a transformation event. The expression of the nucleotide sequence in the expression cassette can be under the control of a constitutive promoter or of an inducible promoter that initiates transcription only when the host cell is exposed to some particular external stimulus. In the case of a multicellular organism such as a plant, the promoter can also be specific to a particular tissue, organ, or stage of development.

As used herein, the term “gene” refers broadly to any segment of DNA associated with a biological function. A gene encompasses sequences including but not limited to a coding sequence, a promoter region, a cis-regulatory sequence, a non-expressed DNA segment is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

The term “gene expression” as used herein refers to the cellular processes by which a biologically active polypeptide is produced from a DNA sequence.

The term “germination” as used herein refers to the process whereby growth emerges in a seed from a period of dormancy. Germination typically involves the proper levels of water, oxygen, temperature, and the proper supporting media to begin growth. In some embodiments, the presently disclosed methods do not involve a germination phase, and hence the time period of the entire process from immature seed to transformation targets can be remarkably concise. To elaborate, using conventional methods, transformation processes require germinating the seeds and preparing the target explants from the germinating seedlings. However, using the presently disclosed methods, soybeans can be transformed without the germination step. Thus, soybean seeds can be isolated from pods and used directly in accordance with the presently disclosed methods. In some embodiments, the seeds can be stored under refrigeration and later used directly in accordance with the presently disclosed methods. The novel method is practical and efficient, and shortens the time of target isolation by eliminating the germination step required by conventional plant transformation methods.

As used herein, the terms “heterologous”, “recombinant”, and “exogenous”, when used herein to refer to a nucleic acid sequence (e.g., a DNA sequence) or a gene, refer to a sequence that originates from a source foreign to the particular host cell or, if from the same source, is modified from its original form. Thus, a heterologous gene in a host cell includes a gene that is endogenous to the particular host cell but has been modified through methods including, but not limited to, the use of DNA shuffling or other recombinant techniques (such as but not limited to cloning the gene into a vector). The terms also include non-naturally occurring multiple copies of a naturally occurring DNA sequence. Thus, the terms refer to a DNA segment that is foreign or heterologous to the cell, or homologous to the cell but in a position or form within the host cell in which the element is not ordinarily found. Similarly, when used in the context of a polypeptide or amino acid sequence, an exogenous polypeptide or amino acid sequence is a polypeptide or amino acid sequence that originates from a source foreign to the particular host cell or, if from the same source, is modified from its original form. Thus, exogenous DNA segments can be expressed to yield exogenous polypeptides.

Accordingly, a polynucleotide sequence is “heterologous to” a second polynucleotide sequence if it originates from a foreign species, or, if from the same species, is modified by human action from its original form. For example, a promoter operably linked to a heterologous coding sequence refers to a coding sequence from a species different from that from which the promoter was derived, or, if from the same species, a coding sequence which is different from any naturally occurring allelic variants.

The term “hypocotyl” as used herein refers to the portion of a plant embryo or seedling located below the cotyledons but above the radicle. A hypocotyl is the portion of an embryo or seedling between the cotyledons and the root. Therefore, it can be considered a transition zone between shoot and root, and typically includes totipotent tissue. In some embodiments, the term a “hypocotyl region” refers to the region of up to about 0.5 cm below a cotyledonary node.

As used herein, the term “immature soybean seed” can refer to a soybean seed at stage late R5 to stage R7.

As used herein, the term “organogenesis” refers to the series of organized integrated processes that transform an amorphous mass of cells into a complete organ (i.e., leaf, etc.) in a developing plant. The cells of an organ-forming region undergo differential development to form an organ primordium. Organogenesis continues until the definitive characteristics of the organ are achieved.

As used herein, the phrase “operatively linked” means that an enhancer-promoter is connected to a coding sequence in such a way that the transcription of that coding sequence is controlled and regulated by that enhancer-promoter. Techniques for operatively linking an enhancer-promoter to a coding sequence are well known in the art; the precise orientation and location relative to a coding sequence of interest is dependent, inter alia, upon the specific nature of the enhancer-promoter.

As used herein, the term “plumule” refers to the primary bud of a plant embryo which can be situated at the apex of the embryo axis, and can include apical meristem, apical region, primary leaf node, axillary meristems, primary leaves and an epicotyl.

The term “pod” as used herein refers to the fruit of a soybean plant. It includes the hull or shell (pericarp) and the soybean seeds.

The term “promoter region” defines a nucleotide sequence within a gene that is positioned 5′ to a coding sequence of a same gene and functions to direct transcription of the coding sequence. The promoter region includes a transcriptional start site and at least one cis-regulatory element. A “functional portion” of a promoter gene fragment is a nucleotide sequence within a promoter region that is required for normal gene transcription. To determine nucleotide sequences that are functional, the expression of a reporter gene is assayed when variably placed under the direction of a promoter region fragment.

The terms “reporter gene” or “marker gene” or “selectable marker” each refer to a heterologous gene encoding a product that is readily observed and/or quantitated. A reporter gene is heterologous in that it originates from a source foreign to an intended host cell or, if from the same source, is modified from its original form. Non-limiting examples of detectable reporter genes that can be operably linked to a transcriptional regulatory region can be found in Alam & Cook (1990) Anal Biochem 188:245-254 and PCT International Publication No. WO 97/47763. Non-limiting examples of reporter genes suitable for transcriptional analyses include the lacZ gene (See, e.g., Rose & Botstein (1983) Meth Enzymol 101:167-180), Green Fluorescent Protein (GFP) (Cubitt et al. (1995) Trends Biochem Sci 20:448-455), luciferase, or chloramphenicol acetyl transferase (CAT). Any suitable reporter and detection method can be used in accordance with the presently disclosed methods, and it can be appreciated by one of skill in the art that no particular choice is essential to or a limitation of the presently disclosed subject matter.

“Shoot” as used herein refers to the aerial portions of the plant and includes the stem, leaves, axillary meristems and apical meristem.

The term “stem apex” as used herein refers to a developmentally important part of a plant that can control branching, direction, leaf and flower production.

As used herein, the term “totipotent” refers to a capacity to grow and develop into a normal plant. Totipotent plant tissue has both the complete genetic information of a plant and the ready capacity to develop into a complete plant if cultured under favorable conditions.

As used herein, the term “transcription factor” refers to a cytoplasmic or nuclear protein that binds to a gene, or binds to an RNA transcript of a gene, or binds to another protein which binds to such gene or such RNA transcript or another protein which in turn binds to such gene or such RNA transcript, so as to thereby modulate expression of the gene. Such modulation can additionally be achieved by other mechanisms; the essence of “transcription factor for a gene” is that the level of transcription of the gene is altered in some way.

As used herein, the terms “transformed”, “transgenic”, and “recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed,” “non-transgenic”, or “non-recombinant” host refers to a wild-type organism, e.g., a bacterium or plant, that does not contain the heterologous nucleic acid molecule.

### III. NUCLEIC ACID SEQUENCES

The presently disclosed subject matter pertains in some embodiments to novel methods for the stable transformation of soybean cells with nucleic acid sequences of interest and to the regeneration of transgenic soybean plants.

Accordingly, the methods of the presently disclosed subject matter can be employed to express any nucleic acid of interest in soybean plants. A gene of interest can include, but is not limited to, a gene for herbicide resistance, disease resistance, or insect/pest resistance, or can be a selectable or scorable marker, and can comprise a plant-operable promoter, a coding region, and a 3′ terminator region. Further, the foreign nucleic acid can include DNA, RNA, and combinations thereof to be inserted into the plant to produce a transformant. In some embodiments, the foreign nucleic acid comprises one or more genes that are contained in a plasmid. Plasmids containing heterologous nucleic acid are available commercially, or can be created in vitro using conventional methods of recombinant DNA manipulation. The plasmid can then be introduced into the vector using conventional methods. The specific nucleic acid can be selected according to the desired properties of the transformant.

Herbicide resistance genes suitable for use in conjunction with the disclosed methods can include, but are not limited to, the AHAS gene for resistance to imidazolinone or sulfonyl urea herbicides, the pat or bar gene for resistance to bialaphos or glufosinate, the EPSP synthase gene for resistance to glyphosate, and so forth. Disease resistance genes can include, but are not limited to, genes for antibiotic synthetic enzymes, e.g., for pyrrolnitrin synthetic enzymes, plant derived resistance genes, and the like. Insect resistance genes can include, but are not limited to, genes for insecticidal proteins from Bacillus thuringiensis and the like. Genes of interest can also encode enzymes involved in biochemical pathways, the expression of which alters a trait that is important in food, feed, nutraceutical, and/or pharmaceutical production.

In accordance with the presently disclosed subject matter, the nucleic acid to be transferred can be contained within an expression cassette. The expression cassette can comprise a transcriptional initiation region linked to a nucleic acid or gene of interest. Such an expression cassette can be provided with a plurality of restriction sites for insertion of the gene or genes of interest (e.g., one gene of interest, two genes of interest, etc.) to be under the transcriptional regulation of the regulatory regions. In some embodiments of the presently disclosed subject matter, the nucleic acid to be transferred contains two or more expression cassettes, each of which encodes at least one gene of interest.

The transcriptional initiation region, (e.g., the promoter) can be native or heterologous to the host. Any suitable promoter known in the art can be employed according to the presently disclosed subject matter (including bacterial, yeast, fungal, insect, mammalian, and plant promoters). Exemplary promoters include, but are not limited to, the Cauliflower Mosaic Virus 35S promoter, the opine synthetase promoters (e.g., nos, mas, ocs, etc.), the ubiquitin promoter, the actin promoter, the ribulose bisphosphate (RubP) carboxylase small subunit promoter, and the alcohol dehydrogenase promoter. Other promoters from viruses that infect plants can also be suitable in the presently disclosed methods including, but not limited to, promoters isolated from Dasheen mosaic virus, Chlorella virus (e.g., the Chlorella virus adenine methyltransferase promoter), tomato spotted wilt virus, tobacco rattle virus, tobacco necrosis virus, tobacco ring spot virus, tomato ring spot virus, cucumber mosaic virus, peanut stump virus, alfalfa mosaic virus, and the like.

As would be understood by one of ordinary skill in the art upon a review of the present disclosure, promoters can be chosen to give a desired level of regulation. For example, in some instances, it can be advantageous to use a promoter that confers constitutive expression (e.g, the ubiquitin promoter, the RubP carboxylase gene family promoters, or the actin gene family promoters). Alternatively, in some embodiments, it can be advantageous to use promoters that are activated in response to specific environmental stimuli (e.g., heat shock gene promoters, drought-inducible gene promoters, pathogen-inducible gene promoters, wound-inducible gene promoters, and light/dark-inducible gene promoters) or plant growth regulators (e.g., promoters from genes induced by abscissic acid, auxins, cytokinins, and gibberellic acid). In some embodiments, promoters can be chosen that give tissue-specific expression (e.g., root, leaf and floral-specific promoters).

The transcriptional cassette can comprise in the 5′-3′ direction of transcription, a transcriptional and translational initiation region, a nucleotide sequence of interest, and a transcriptional and translational termination region functional in plants. Any suitable termination sequence known in the art can be used in accordance with the presently disclosed subject matter. The termination region can be native to the transcriptional initiation region, native to the nucleotide sequence of interest, or can be derived from another source. In some embodiments, termination regions can be used from the Ti-plasmid of Agrobacterium tumefaciens, such as the octopine synthetase and nopaline synthetase termination regions. See, Guerineau et al. (1991) Mol. Gen. Genet. 262: 141; Proudfoot (1991) Cell 64: 671; Sanfacon et al. (1991) Genes Dev. 5:141; Mogen et al. (1990) Plant Cell 2:1261; Munroe et al. (1990) Gene 91: 151; Ballas et al. (1989) Nucleic Acids Res. 17: 7891; and Joshi et al. (1987) Nucleic Acids Res. 15: 9627. Additional termination sequences that can be used in the presently disclosed subject matter are the pea RubP carboxylase small subunit termination sequence and the Cauliflower Mosaic Virus 35S termination sequence. Other suitable termination sequences will be apparent to those of ordinary skill in the art.

Alternatively, in some embodiments, the genes of interest can be provided on any other suitable expression cassette known in the art. Where appropriate, the genes can be optimized for increased expression in the transformed plant. Where mammalian, yeast, bacterial or plant dicot genes are used in the presently disclosed subject matter, they can be synthesized using monocot or soybean preferred codons for improved expression. Methods are available in the art for synthesizing plant preferred genes. See, e.g., U.S. Pat. No. 5,380,831; U.S. Pat. No. 5,436,391; and Murray et al. (1989) Nucleic Acids. Res. 17: 477; herein incorporated by reference in their entireties.

The expression cassettes can additionally contain 5′ leader sequences. Such leader sequences can act to enhance translation. Translation leaders are known in the art and can include, but are not limited to, picornavirus leaders (e.g., EMCV leader), potyvirus leaders, human immunoglobulin heavy-chain binding protein untranslated leader from the coat protein mRNA of alfalfa mosaic virus, tobacco mosaic virus leader, and maize chlorotic mottle virus leader. Other methods known to enhance translation can also be utilized, e.g., introns and the like.

The expression cassettes can contain more than one gene or nucleic acid sequence to be transferred and expressed in the transformed plant. Thus, each nucleic acid sequence can be operably linked to 5′ and 3′ regulatory sequences. Alternatively, multiple expression cassettes can be provided.

In some embodiments, the expression cassette can comprise a selectable marker gene for the selection of transformed cells. Selectable marker genes can be utilized for the selection of transformed cells or tissues. Selectable marker genes can include, but are not limited to, genes encoding antibiotic resistance, such as those encoding neomycin phosphotransferase II (NEO) and hygromycin phosphotransferase (HPT), as well as genes conferring resistance to herbicidal compounds. Herbicide resistance genes can code for a modified target protein insensitive to the herbicide or for an enzyme that degrades or detoxifies the herbicide in the plant before it can act. See, for example, DeBlock et al. (1987) EMBO J. 6: 2513); DeBlock et al. (1989) Plant Physiol. 91: 691; Fromm et al. (1990) Bio Technology 8: 833; Gordon-Kamm et al. (1990) Plant Cell 2: 603. For example, resistance to glyphosphate or sulfonylurea herbicides can been obtained using genes coding for the mutant target enzymes 5-enolpyruvylshikimate-3-phosphate synthase and acetolactate synthase. Further, resistance to glufosinate ammonium, boromoxynil, and 2,4-dichlorophenoxyacetate (2,4-D) can be accomplished by using bacterial genes encoding phosphinothricin acetyltransferase, a nitrilase, or a 2,4-dichlorophenoxyacetate monooxygenase, which detoxify the respective herbicides.

For purposes of the presently disclosed subject matter, selectable marker genes include, but are not limited to, genes encoding: gentamicin, kanamycin, streptomycin, azithromycin, erythromycin, penicillin G, penicillin V, oxacillin, cloxacillin, dicloxacillin, ampicillin, amoxicillin, ticarcillin, ciprofloxacin, doxycycline, minocycline, tetracycline, glyphosate, sulfonylurea, imidazolinone, glufosinate, phenoxy proprionic acid, cycloshexone, triazine, benzonitrile, and combinations thereof.

The bar gene confers herbicide resistance to glufosinate-type herbicides, such as phosphinothricin or bialaphos, and the like. As noted above, other selectable markers that could be used in the vector constructs include, but are not limited to, the pat gene, also for bialaphos and phosphinothricin resistance, the ALS gene for imidazolinone resistance, the HPH or HYG gene for hygromycin resistance, the EPSP synthase gene for glyphosate resistance, the Hml gene for resistance to the Hc-toxin, and other selective agents used routinely and known to one of ordinary skill in the art. See, for example, Yarranton (1992) Curr. Opin. Biotech 3: 506 (1992); Yao et al. (1992) Cell 71: 63; Reznikoff (1992) Mol. Microbiol. 6: 2419; Hu et al. (1987) Cell 48, 555; Brown et al. (1987) Cell 49: 603; Figge et al. (1988) Cell 52: 713; Deuschle et al. (1989) Proc. Natl. Acad. Sci. USA 86: 5400; Fuerst et al. (1989) Proc. Natl. Acad. Sci. USA 86: 2549; Deuschle et al. (1990) Science 248: 480, herein incorporated by reference. The above list of selectable marker genes are not meant to be limiting, and any selectable marker gene can be used in the presently disclosed subject matter.

Where appropriate, the selectable marker genes and other genes and nucleic acids of interest to be transferred can be synthesized for optimal expression in soybean cells. Particularly, the coding sequence of the genes can be modified to enhance expression in soybean cells. The synthetic nucleic acid can be designed to be expressed in the transformed tissues and plants at a higher level. Accordingly, the use of optimized selectable marker genes can result in higher transformation efficiency.

Additional sequence modifications are known in the art to enhance gene expression in a cellular host. Particularly, sequences encoding spurious polyadenylation signals, exon-intron splice site signals, transposon-like repeats, and other such well-characterized sequences that can be deleterious to gene expression can be eliminated. In some embodiments, the G-C content of the sequence can be adjusted to levels average for a given cellular host, as calculated by reference to known genes expressed in the host cell. When desired, the sequence can be modified to avoid predicted hairpin secondary mRNA structures.

### IV. TARGET TISSUES

The starting material for the transformation methods disclosed herein is an immature soybean seed, which can be isolated from a growing soybean plant. In some embodiments, the immature soybean seeds are isolated from greenhouse-grown soybean plants. As would be apparent to one of ordinary skill in the art, the soybean plants can be grown using standard conditions for the successful growth of plants, i.e. grown under about 16 hours of daylight at about 24° C. Soybean pods can be isolated from the plants using standard techniques, including but not limited to, removal of the pod using a gloved hand, removal using any of a number of mechanical devices, and the like. Further, removal of the soybean seeds from the pods can be accomplished using standard techniques, including but not limited to, using a sterilized gloved hand, using any of a number of mechanical devices, and the like. The immature soybean seeds can be sterilized after removal from the pods using standard techniques, including but not limited to, rinsing with water, diluted Clorox bleach, and/or alcohol one or more times. The immature seeds can be used immediately after removing from the pods, or can be stored for later use.

As would be readily understood by one of skill in the art, seed storage can be accomplished by any of a variety of known methods. For example, the seeds can be stored at 4° C. for later use.

The immature soybean seeds suitable for use in the presently disclosed methods can comprise soybean seeds isolated from the plant at late R5 to R7 phase. As would be readily understood by one of skill in the art, R5 refers to the phase of beginning seed, R6 refers to full seed phase, and R7 refers to beginning maturity phase.

To elaborate, a germination step is required prior to transformation when using mature dry soybean seeds in the conventional methods. The presently disclosed method does not involve a germination phase, and hence the time period for transformation target explant preparation is shortened. Thus, soybean seeds can be isolated and used directly in accordance with the presently disclosed methods. In addition, this novel method also increases the percentage of useable seeds due to the absence of fungal contamination compared to using sterilized dry seeds.

Soybean explants can be prepared from the immature soybean seeds using any of a variety of disclosed methods. Particularly, in some embodiments, explants can be prepared from the immature soybean seeds by removing the seed coat, one cotyledon, and two primary leaves. In some embodiments, the plumule including the apical meristem can then be wounded. The cotyledons are the first pair of leaves of the seed, and the primary leaves are the leaves that are subsequently produced by the plant when the stem or shoot grows.

In some embodiments, explants can be prepared from the immature soybean seeds by removing the hypocotyl region up to about 0.5 cm below the cotyledonary node, and removing the seed coat. In some embodiments, one cotyledon and two primary leaves can further be removed. In some embodiments, the cotyledonary node region and the plumule including the apical meristem can be wounded.

In some embodiments, explants can be prepared from the immature soybean seeds by separating the seed into two halves. In some embodiments, the seed is separated longitudinally in the middle of the embryo axis, such that both halves of the resulting explant include one cotyledon and part of the immature embryo axis. In some embodiments, both primary leaves can be removed and the apical and/or cotyledonary node region of the seed wounded.

In some embodiments, explants can be prepared from the immature soybean seeds by removing the seed coat, and wounding the plumule or cotyledonary node region.

In some embodiments, explants can be prepared from the immature soybean seeds by removing the seed coat and part of the two cotyledons and primary leaves.

In some embodiments, explants can be prepared from the immature soybean seeds by removing all or part of the two cotyledons and two primary leaves, and wounding the apical and/or cotyledonary node meristem.

As indicated hereinabove, preparing the explant can comprise wounding the explant in some embodiments. As would be readily understood by one of skill in the art, wounding can comprise any injury to the tissue of the explant. In some embodiments, the wounding can comprise one or more cuts, stabs, lacerations, lesions or traumas inflicted to the tissue of an explant. In some embodiments, the wounding can be inflicted by a mechanical instrument (such as but not limited to a scalpel blade).

As indicated hereinabove, preparing the explant can comprise removing portions of the explant in some embodiments. As would be readily understood by one of skill in the art, removing can comprise extracting all or part of the indicated plant organ. For example, “removing the seed coat” can comprise removing all of the seed coat, or removing any of a portion of the seed coat.

### V. AGROBACTERIUM-MEDIATED TRANSFORMATION

Agrobacterium-mediated gene transfer exploits the natural ability of Agrobacterium to transfer DNA into plant chromosomes. As is well-known in the art, Agrobacterium is a plant pathogen that can transfer a set of genes into plant cells. In some embodiments of the presently disclosed subject matter, immature soybean cells can be transformed using Agrobacterium tumefaciens.

Those skilled in the art will appreciate that the disclosed methods apply equally well to Agrobacterium rhizogenes. Transformation using Agrobacterium rhizogenes has developed analogously to that of Agrobacterium tumefaciens and has been successfully utilized to transform plants, including but not limited to, alfalfa, Solanum nigrum L., and poplar. See, for example, Hooykaas, Plant Mol. Biol. (1989) 13: 327; Smith et al., Crop Science (1995) 35: 301 (1995); Chilton, Proc. Natl. Acad. Sci. USA (1993) 90: 3119; Mollony et al., Monograph Theor. Appl. Genet NY(1993) 19: 148; Ishida et al. Nature Biotechnol. (1996) 14: 745 (1996); and Komari et al., The Plant Journal (1996) 10:165 (1996), the disclosures of which are incorporated herein by reference.

For Agrobacterium-mediated gene transfer, wounding of the explant tissue can be used to facilitate gene transfer. Accordingly, in some embodiments, a wound can be created in the explant tissue.

The Agrobacterium-mediated transformation process of the presently disclosed subject matter can comprise several steps. The basic steps can include but are not limited to, an infection step and a co-cultivation step. In some embodiments, these steps are followed by a selection step, and in some embodiments by a selection and a regeneration step, as discussed in detail hereinbelow.

In the infection step, the soybean cells to be transformed are exposed to Agrobacterium. In some embodiments, the cells are brought into contact with the Agrobacterium in a liquid medium. In some embodiments, the Agrobacterium can be modified to contain a gene or nucleic acid of interest, wherein the nucleic acid can be inserted into the vector. General molecular biology techniques used in the presently disclosed subject matter are well-known by those of skill in the art.

Agrobacterium containing a plasmid of interest can be maintained on Agrobacterium master plates with stock frozen at about −80° C. Master plates can then be used to inoculate agar plates to obtain Agrobacterium that is then resuspended in medium for use in the infection process. Alternatively, bacteria from the master plate can be used to inoculate broth cultures that are grown to logarithmic phase prior to transformation.

The concentration of Agrobacterium used in the infection step and co-cultivation step can affect the transformation frequency. Thus, the concentration of Agrobacterium useful in the methods of the presently disclosed subject matter can vary depending on the Agrobacterium strain utilized, the tissue being transformed, the soybean species being transformed, and the like. To optimize the transformation protocol for a particular soybean species or tissue, the tissue to be transformed can be incubated with various concentrations of Agrobacterium. Likewise, the level of marker gene expression and the transformation efficiency can be assessed for various Agrobacterium concentrations. While the concentration of Agrobacterium can vary, generally a concentration range of about 1×10³ cfu/ml to about 1×10¹⁰ cfu/ml can be employed in the methods of the presently disclosed subject matter. In some embodiments, the concentration of Agrobacterium can vary from about 1×10³ cfu/ml to about 1×10⁹ cfu/ml. In some embodiments, the concentration of Agrobacterium can vary from about 1×10⁸ to about 1×10⁹.

The soybean tissue to be transformed can generally be added to the Agrobacterium suspension in a liquid contact phase containing a concentration of Agrobacterium to optimize transformation efficiencies. The contact phase facilitates maximum contact of the tissue to be transformed with the suspension of Agrobacterium. Infection generally can be allowed to proceed for up to about 24 hours.

Those skilled in the art will appreciate that the conditions can be optimized to achieve the highest level of infection and transformation by Agrobacterium. In some embodiments, one or more virulence-enhancing compounds (such as, but not limited to, acetosyringone) can be added to enhance gene delivery. Furthermore, to enhance transformation frequency, in some embodiments, tissue can be cultured in medium containing antioxidants including but not limited to cysteine. As further alternatives, tissue wounding, vacuum pressure, or cultivation in medium containing acetosyringone can be employed to promote the transformation efficiency.

For Agrobacterium-mediated transformation, the immature embryos can be co-cultured for a time with the Agrobacterium in order to increase transformation efficiency. In the co-cultivation step, the majority of the Agrobacterium cells are removed by pouring or pipetting, and the explants are co-cultivated with the remainder of the Agrobacterium. Particularly, in the co-cultivation step, the soybean explants can be co-cultivated with Agrobacterium on a co-cultivation medium (such as but not limited to SoyCCM, SoyCoC, and the like). In some embodiments, the soybean explants can be co-cultivated with the Agrobacterium for up to about 7 days. Co-cultivation can be carried out in the dark or under light conditions in some embodiments to enhance the transformation efficiency. Additionally, as described herein above for the inoculation step, co-culturing can be done on medium containing acetosyringone to promote transformation efficiency. In some embodiments, the co-culturing step can be performed in the presence of cytokinins, which can act to enhance cell proliferation.

In some embodiments, after the co-cultivation step, excess bacteria are removed from the explants by washing in a solution containing antibiotics. In some embodiments, the excess bacteria are removed by blotting with filter paper, washing, decanting excess bacteria, and the like.

Following the co-cultivation step, or resting/decontamination step, transformants can be selected and soybean plants regenerated as described herein below.

### VI. TRANSFORMATION OF IMMATURE SOYBEAN SEEDS BY BALLISTIC BOMBARDMENT

In some embodiments, the presently disclosed subject matter comprises a method of transforming immature soybean seeds with a nucleotide sequence of interest using a microprojectile.

According to some embodiments of the presently disclosed subject matter, the ballistic transformation method comprises the steps of providing a soybean immature seed tissue as a target, and propelling the microprojectile carrying the nucleotide sequence at the soybean tissue at a velocity sufficient to pierce the walls of the cells within the tissue and to deposit the nucleotide sequence within a cell of the tissue to thereby provide a transformed tissue. In some embodiments of the presently disclosed subject matter, the method further includes culturing the transformed tissue with a selection agent, as described herein below. In some embodiments, the selection step is followed by the step of regenerating transformed soybean plants from the transformed tissue.

As would be apparent to one of skill in the art, any ballistic cell transformation apparatus can be used in practicing the presently disclosed subject matter. See, for example, Sandford et al. (Particulate Science and Technology (1988) 5:27), Klein et al. (Nature (1987) 327:70), and in European Patent Application No. EP 270,356.

In some embodiments, a commercially-available helium gene gun (PDS-1000/He) manufactured by DuPont (Wilmington, Del., United States of America) can be employed. Alternately, an apparatus configured as described by Klein et al. (Nature (1987) 327:70) can be utilized, comprising, in some embodiments, a bombardment chamber, which is divided into two separate compartments by an adjustable-height stopping plate.

The microprojectile can be formed from any material having sufficient density and cohesiveness to be propelled through the cell wall, given the particle's velocity and the distance the particle must travel. Examples of materials suitable for making microprojectiles include, but are not limited to, metal, glass, silica, ice, polyethylene, polypropylene, polycarbonate, and carbon compounds (e.g., graphite, diamond). The particles should be of a size sufficiently small to avoid excessive disruption of the cells they contact in the target tissue, and sufficiently large to provide the inertia required to penetrate to the cell of interest in the target tissue. Particles ranging in diameter from about one-half micrometer to about three micrometers are suitable. Particles need not be spherical, as surface irregularities on the particles can enhance their DNA carrying capacity.

In some embodiments, the nucleotide sequence can be immobilized on the particle by precipitation. The precise precipitation parameters employed can vary depending upon factors such as the particle acceleration procedure employed, as is well known in the art. The carrier particles can optionally be coated with an encapsulating agents such as polylysine to improve the stability of nucleotide sequences immobilized thereon.

In some embodiments, ballistic transformation is achieved without use of microprojectiles. For example, an aqueous solution containing the nucleotide sequence of interest as a precipitate can be carried by the macroprojectile (e.g., by placing the aqueous solution directly on the plate-contact end of the macroprojectile without a microprojectile, where it is held by surface tension), and the solution alone propelled at the plant tissue target (e.g., by propelling the macroprojectile down the acceleration tube in the same manner as described hereinabove). Other approaches include placing the nucleic acid precipitate itself (“wet” precipitate) or a freeze-dried nucleotide precipitate directly on the plate-contact end of the macroprojectile without a microprojectile. In some embodiments, the nucleotide sequence can be propelled at the tissue target in the absence of a microprojectile.

After ballistic bombardment of the target tissue, transformants can be selected and soybean plants regenerated as described hereinbelow.

### VII. POST-TRANSFORMATION EXPLANT GROWTH

As discussed in detail hereinabove, soybean tissue can be transformed according to the presently disclosed subject matter (including but not limited to ballistic bombardment or Agrobacterium-mediated transformation). After the transformation step, the transformed tissue can be exposed to selective pressure to select for those cells that have received and are expressing the polypeptide from the heterologous nucleic acid introduced by the expression cassette. The agent used to select for transformants can select for preferential growth of cells containing at least one selectable marker insert positioned within the expression cassette and delivered by ballistic bombardment or by Agrobacterium.

In some embodiments, the transformed tissue can be subjected to an optional resting and decontamination step. For the resting/decontamination step, the transformed cells can be transferred to a medium in the absence of any selective pressures to permit recovery and proliferation of transformed cells containing the heterologous nucleic acid.

The resting/decontamination step can be carried out for as long as is necessary to inhibit the growth of Agrobacterium and to increase the number of transformed cells prior to selection. In some embodiments, the resting/decontamination step can be carried out for up to about 2 weeks. In some embodiments, after explants are transformed by Agrobacterium-mediated methods, the explants can be transferred to recovery medium, such as but not limited to SoyR1, to induce growth. In some embodiments, following the co-cultivation step, elongated hypocotyls of the explants can be trimmed back below the cotyledon nodes, and the transformed tissue subjected to an optional resting and decontamination step. For the resting/decontamination step, the transformed cells can be transferred to a recovery medium, such as but not limited to SoyR1. In some embodiments, the resting phase is performed in the absence of any selective pressures to permit recovery and proliferation of transformed cells containing the heterologous nucleic acid. In some embodiments, an antibiotic is added to the recovery medium to kill or inhibit Agrobacterium growth. Representative antibiotics are known in the art, including but not limited to, cefotaxime, timetin, vancomycin, carbenicillin, gentamicin, kanamycin, streptomycin, azithromycin, erythromycin, penicillin G, penicillin V, oxacillin, cloxacillin, dicloxacillin, ampicillin, amoxicillin, ticarcillin, ciprofloxacin, doxycycline, minocycline, tetracycline, and the like. Concentrations of the antibiotic can vary according to what is standard for each antibiotic. For example, concentrations of carbenicillin can range from about 50 mg/l to about 250 mg/l carbenicillin in solid media. Those of ordinary skill in the art will recognize that the concentration of antibiotic can be optimized for a particular transformation protocol without undue experimentation.

In some embodiments, the resting phase cultures can be maintained under a 16 hour light-8 hour dark regimen. In some embodiments, the explants can be incubated under light intensity of up to about 160 μE/m²/s. The recovery step can be carried out for as long as is necessary to inhibit the growth of Agrobacterium and to increase the number of transformed cells prior to selection. In some embodiments, the recovery step can be carried out for 1 to 3 weeks. In some embodiments, the recovery step can be carried out for 1 to 2 weeks. In some embodiments, the recovery step can be carried out for about 1 week. In some embodiments, the entire recovery step is omitted. In some embodiments, the recovery medium can contain a shoot-inducing hormone (such as but not limited to zeatin). The medium can also comprise cell growth regulating compounds that enhance cell proliferation and induce shoot formation, including but not limited to, auxins (such as but not limited to IAA, NAA, and IBA), cytokinins (such as but not limited to thidiazuron, kinetin, BAP, Zeatin, and isopentenyl adenine) and/or gibberellic acids (GA₃).

The conditions under which selection for transformants can be performed can represent an aspect of the methods disclosed herein. As would be apparent to one of skill in the art upon a review of the present disclosure, the transformation process subjects the cells to stress, and the selection process can be toxic even to transformants. In some embodiments, in response to this concern, the transformed tissue can be initially subjected to weak selection, utilizing low concentrations of the selection agent and subdued light, with a gradual increase in the applied selection gradient by increasing the concentration of the selection agent and/or increasing the light intensity. In some embodiments, selection pressure can be removed altogether for a period of time and then reapplied if the tissue becomes stressed. In some embodiments, the selection medium can contain a simple carbohydrate, such as but not limited to 1% to 3% sucrose to ensure that the cells do not carry out photosynthesis. In addition, the selection can be initially performed under subdued light conditions, or even in complete darkness, so as to keep the metabolic activity of the cells at a relatively low level. Those skilled in the art can appreciate that the specific conditions under which selection is performed can be optimized for every species or strain of soybean without undue experimentation.

As would be appreciated by one of skill in the art upon a review of the present disclosure, selection can be carried out long enough to kill non-transformants and to allow transformed cells to proliferate at a similar rate to non-transformed cells. Thus, in some embodiments, the selection period can be longer with cells that proliferate at a slower rate.

Regeneration is the process whereby an organism restores or grows organs, tissues, etc. that have been lost, removed, and/or injured. In some embodiments of the presently disclosed subject matter, regeneration can refer to the process of growing a plant from a plant cell (such as but not limited to an explant). As used herein, regeneration is described in several steps (e.g., elongation, growth, rooting) for convenience, not by way of limitation.

Selection can be pursued in a medium suitable to initiate regeneration of a plant, i.e., a “regeneration medium.” In some embodiments, the regeneration medium comprises a relatively high concentration of selection agent when compared to the recovery medium. For example, in some embodiments, the regeneration medium can comprise about 2-8 mg/L glufosinate for selection.

In some embodiments, the regeneration medium can contain a shoot-inducing hormone (such as but not limited to BAP). The medium can also comprise cell growth regulating compounds that induce shoot formation, including but not limited to, auxins (such as but not limited to IAA, NAA, and IBA), cytokinins (such as but not limited to thidiazuron, kinetin, and isopentenyl adenine) and/or gibberellic acids (GA₃).

In some embodiments, one explant in recovering media can be transferred as one unit to regeneration medium to induce growth. In some embodiments, the explant in recovery medium can be separated into several pieces and placed in regeneration medium to induce growth. For example, elongated epicotyls and/or developing shoots can be excised and transferred along with the cotyledons to regeneration medium (such as but not limited to SoyR2) to induce growth.

In some embodiments, the explants can remain in regeneration medium for about 1 to 3 weeks. In some embodiments, the explants can remain in regeneration medium for about 1-2 weeks. In some embodiments, the explants can remain in regeneration medium for about 2 weeks.

After sufficient time in regeneration medium, developed or developing explant shoots can be excised and transferred to an elongation medium (such as but not limited to SoyE1) for shoot elongation. In some embodiments, the elongation medium comprises a selection agent, such as but not limited to 0.1-10 mg/L glufosinate. In some embodiments, the elongation medium does not include a selection agent.

In some embodiments, the elongation medium can contain a shoot-inducing hormone (such as but not limited to zeatin). The medium can also comprise cell growth regulating compounds that enhance cell proliferation and induce shoot elongation, including but not limited to, auxins (such as but not limited to IAA, NAA, and IBA), cytokinins (such as but not limited to thidiazuron, kinetin, BAP, zeatin, and isopentenyl adenine) and/or gibberellic acids (GA₃).

In some embodiments, subculture to fresh elongation medium can be performed about every two weeks. In some embodiments, elongated shoots can then be transferred to an elongation medium with reduced amount of hormone (such as but not limited to SoyE2) to enhance shoot elongation. In some embodiments, when the shoots reach about 2 cm or more and/or have full leaf formation, they can be separated from the explant and transferred to the rooting medium.

After a sufficient time in elongation medium, shoots can be transferred to a rooting medium (such as but not limited to SoyRoot) to induce root growth. In some embodiments, the rooting medium can contain a root-inducing hormone, as are known in the art. In some embodiments, the rooting medium can comprise a selection agent to further help identify potential transformed shoots.

During the regeneration process, any method known in the art can be utilized to verify that the regenerating plants are transformed with the transferred nucleic acid of interest. For example, histochemical staining, ELISA assay, Southern hybridization, Northern hybridization, Western immunoblotting, PCR, Taqman assay, and the like can be used to detect the transferred nucleic acids or protein in the regenerating plants. In some embodiments, leaves can be sampled for analysis to identify transformants. Particularly, a portion of the plant sample can be assayed for the presence of the foreign nucleic acid or the protein that such nucleic acid encodes. Positives are rooted and transplanted to soil and grown in greenhouse to fully mature and for seeds.

### VIII. TRANSGENIC PLANTS AND SEEDS

Transgenic plants comprising a heterologous nucleic acid (i.e., comprising cells or tissues transformed in accordance with the methods described herein), as well as the seeds and progeny produced by the transgenic plants, are an additional aspect of the presently disclosed subject matter. Procedures for cultivating transformed cells to useful cultivars are known to those skilled in the art. Techniques are known for the in vitro culture of plant tissue, and in a number of cases, for regeneration into whole plants. In some embodiments, the presently disclosed subject matter comprises transgenic plant tissue, plants, or seeds containing the nucleic acids described above.

As provided hereinabove, seeds and progeny plants of the regenerated plants can comprise an aspect of the presently disclosed subject matter. Accordingly, the term “seeds” can encompass seeds of the transformed plant, as well as seeds produced from the progeny of the transformed plants. Plants of the presently disclosed subject matter can include not only the transformed and regenerated plants, but also progeny of transformed and regenerated plants produced by the methods described herein.

Plants produced by the described methods can be screened for successful transformation by standard methods described above. Seeds and progeny plants of regenerated plants of the presently disclosed subject matter can be continuously screened and selected for the continued presence of the transgenic and integrated nucleic acid sequence in order to develop improved plant and seed lines, which are another aspect of the presently disclosed subject matter. Desirable transgenic nucleic acid sequences can thus be moved (i.e., introgressed or inbred) into other genetic lines such as certain elite or commercially valuable lines or varieties. Methods of introgressing desirable nucleic acid sequences into genetic plant lines can be carried out by a variety of techniques known in the art, including by classical breeding, protoplast fusion, nuclear transfer and chromosome transfer. Breeding approaches and techniques are known in the art, and are set forth in, for example, J. R. Welsh, Fundamentals of Plant Genetics and Breeding (John Wiley and Sons, New York, (1981)); Crop Breeding (D. R. Wood, ed., American Society of Agronomy, Madison, Wis., (1983)); O. Mayo, The Theory of Plant Breeding, Second Edition (Clarendon Press, Oxford, England (1987)); and Wricke and Weber, Quantitative Genetics and Selection Plant Breeding (Walter de Gruyter and Co., Berlin (1986)). Using these and other techniques in the art, transgenic plants and inbred lines obtained according to the presently disclosed subject matter can be used to produce commercially valuable hybrid plants and crops, which hybrids are also an aspect of the presently disclosed subject matter.

### EXAMPLES

The following Examples have been included to illustrate representative and exemplary modes of the presently disclosed subject matter. In light of the present disclosure and the general level of skill in the art, those of skill will appreciate that the following Examples are intended to be exemplary only and that numerous changes, modifications, and alterations can be employed without departing from the spirit and scope of the presently disclosed subject matter.

### Materials and Methods

The following media were used in the Examples described herein.

### SoyInf Medium

2.15 g MS basal salt mixture, 5 ml B5 vitamins 200×, 20 g sucrose, 10 g glucose, 4 g MES, and 2 ml Zeatin riboside trans isomers (from a 1 mg/ml stock solution) were combined and taken up to a final volume of 1 L using sterile water. The pH was adjusted to 5.4.

### SoyCCM Medium

20 g sucrose, 4 g MES, 6 g purified agar, 990 ml Evian water, 1 ml acetosyringone (from a 40 mg/ml stock solution) and 0.5 ml BAP (from a 1 mg/ml stock solution) were combined and taken to a final volume of 1 L using sterile water. The pH was adjusted to 5.4.

### SoyCoC Medium

2.15 g MS basal salt mixture, 5 ml B5 vitamins 200×, 20 g sucrose, 10 g glucose, 4 g MES, 2 ml Zeatin riboside trans isomers (from a 1 mg/ml stock solution), and 6 g purified Agar were combined and taken up to a final volume of 1 L using sterile water. The pH was adjusted to 5.4.

### SoyR1 Medium

3.1 g B5 basal salt (Gamborg's), 4 ml B5 vitamins 200×, 4 ml MS Iron 200×, 100 mg asparagine, 10 ml MES (from a 100 mg/ml stock solution), 2 ml Zeatin riboside trans isomers (from a 1 mg/ml stock solution), 30 g sucrose, 7 g purified agar, 2 ml glutamine (from a stock 50 mg/ml solution), and 3 ml ticarcillin:potassium clavulanate 15:1 (from a 100 mg/ml stock solution) were combined and taken up to a final volume of 1 L using sterilized water. The pH was adjusted to 5.6.

### SoyR2 Medium

3.1 g B5 basal salt (Gamborg's), 5 ml B5 vitamins 5×, 4 ml MS iron 200×, 100 mg asparagine, 10 ml MES (from a 100 mg/ml stock solution), 1 ml BAP (from a 1 mg/ml stock solution), 30 g sucrose, 7 g purified agar, 2 ml glutamine (from a 50 mg/ml stock solution), and 3 ml Ticarcillin:potassium clavulanate 15:1 (from a 100 mg/ml stock solution) were combined and taken up to a final volume of 1 L using sterilized water. The pH was adjusted to 5.6.

### SoyE1 Medium

4.3 g MS basal salt mixture, 5 ml B5 vitamins 200×, 3 ml MS iron 200×, 30 g sucrose, 590 mg MES, 7 g purified agar, 3 ml Ticarcillin:potassium clavulanate 15:1 (from a 100 mg/ml stock solution), 0.4 ml Cefotaxime (from a 250 mg/ml stock solution), 0.1 ml IAA (from a 1 mg/ml stock solution), 0.1 ml GA3 (from a 5 mg/ml stock solution), 2 ml glutamine (from a stock 50 mg/ml solution), 2 ml asparagine (from a 25 mg/ml stock solution), and 1 ml Zeatin Riboside trans isomers (from a 1 mg/ml stock solution) were combined and taken up to a final volume of 1 L using sterilized water. The pH was adjusted to 5.6.

### SoyE2 Medium

4.3 g MS basal salt mixture, 5 ml B5 vitamins 200×, 3 ml MS iron 200×, 30 g sucrose, 590 mg MES, 3 g Gelrite, 3 ml Ticarcillin:potassium clavulanate 15:1 (from a stock 100 mg/ml solution), 0.4 ml Cefotaxime (from a 250 mg/ml stock solution), 2 ml Glutamine (from a 50 mg/ml stock solution), and 2 ml Asparagine (from a 25 mg/ml stock solution) were taken up to a final volume of 1 L using sterile water. The pH was adjusted to 5.4.

### SoyRoot Medium

2.2 g MS basal salt mixture, 5 ml B5 vitamins 200×, 3 ml MS Iron 200×, 20 g sucrose, 590 mg MES, 3 g Gelrite, 2 ml Glutamine (from a 50 mg/ml stock solution), 2 ml asparagine (from a 25 mg/ml stock solution), and 0.6 ml of IBA (from a 1 mg/ml stock solution) were combined and taken up to a final volume of 1 L using sterile water. The pH was adjusted to 5.4.

### EXAMPLE 1

### Isolation of Seeds from Seed Pods of Different Developing Stages

Soybean (Glycine max cultivars Jack, Williams 82, or S42H1) stock plants were grown in a greenhouse under 16 hours of daylight at 24° C. Pods at developing stages from late R5 to R7 (filled seeds with green to yellow pod color) were collected and sterilized by immersing in 70% ethanol for 30 seconds, or in 10-20% Clorox bleach for 20 minutes. Sterilized pods were then rinsed thoroughly with sterile water.

Seeds were isolated from the sterilized pods using gloved hands sprayed with 70% ethanol. The isolated seeds were rinsed thoroughly with sterile water or further sterilized with 10% Clorox bleach up to ten minutes followed by thorough rinsing with sterile water.

The sterilized seeds were used immediately for preparing explants for transformation, or were stored at 4° C. for later use.

### EXAMPLE 2

### Transformation Vector and Agrobacterium Strains

Binary vectors 15237 (with CMP promoter—bar gene—nos terminator expression cassette) or 15238 (with CMP promoter—bar gene—nos terminator and CMP promoter—ZsGreenFP—nos terminator expression cassette)were used for Agrobacterium-mediated transformation.

The vectors were introduced separately into Agrobacterium tumefaciens strain LB4404 or EHA101 using electroporation. Single bacterial colonies containing each of the vectors were selected to confirm the presence of intact vector and used for further experiments.

### EXAMPLE 3

### Preparation of Agrobacterium for Transformation

Agrobacterium culture was initiated weekly from glycerol stock at −80° C. onto YP semi-solid medium containing appropriate antibiotics and grown at 28° C. in an incubator.

The Agrobacterium was streaked onto fresh YP medium containing appropriate antibiotics the day before the inoculation and was grown in a 28° C. incubator. For plant transformation use, the Agrobacterium was collected from the plate using a disposable plastic inoculation loop and suspended in liquid infection medium, such as SoyInf, in a sterile 50 ml disposable polypropylene centrifugation tube. The tube was shaken gently until the Agrobacterium cells were uniformly dispersed in the suspension. The bacterial cell suspension was then diluted to A₆₆₀ of 0.5 to 0.8, and acetosyringone was added to a final concentration of 40 to 80 mg/L (approximately 200 to 400 μM) to induce virulence gene expression.

### EXAMPLE 4

### Preparation of Transformation Targets

Explants were prepared from sterilized soybean immature seeds isolated directly from pods as described in Example 1 without further germination or culture. Explants were inoculated in Agrobacterium suspension of Example 3 for up to 24 hours. Explant preparation included one of the following procedures, as set forth in Examples 4.1 through 4.9:

### EXAMPLE 4.1

The seed coat was removed from the seed. One cotyledon and two primary leaves were removed.

### EXAMPLE 4.2

The seed coat was removed from the seed. One cotyledon and two primary leaves were removed. Cotyledonary node region and/or plumule including apical meristem were then wounded using the sharp end of a scalpel.

### EXAMPLE 4.3

A hypocotyl region was removed by cutting below the cotyledonary node, and the seed coat was removed. One cotyledon and two primary leaves were removed.

### EXAMPLE 4.4

A hypocotyl region was removed by cutting below the cotyledonary node, and the seed coat was removed. One cotyledon and two primary leaves were removed. Then, cotyledonary node region and/or the plumule including apical meristem were wounded with the sharp end of a scalpel.

### EXAMPLE 4.5

A hypocotyl region was removed by cutting below a cotyledonary node. The hilum was removed and the seed was separated into two halves by cutting longitudinally in the middle of the embryo axis. Both halves of the resulting explant included one cotyledon and part of the immature embryo axis.

### EXAMPLE 4.6

A hypocotyl region was removed by cutting below the cotyledonary node. The hilum was removed and the seed was separated into two halves by cutting longitudinally in the middle of the embryo axis. Both halves of the resulting explant included one cotyledon and part of the immature embryo axis. Both primary leaves were removed.

### EXAMPLE 4.7

A hypocotyl region was removed by cutting below the cotyledonary node. The hilum was removed and the seed is cut longitudinally and into two halves in the middle of an embryo axis. Both primary leaves were removed and the apical region of the plant including the cotyledonary node and plumule was further wounded. Both halves of the resulting explants included one cotyledon and part of the embryo axis.

### EXAMPLE 4.8

The seed coat was removed from the seed. A scalpel blade was inserted between two cotyledons and a plumule was wounded using the sharp end of the scalpel blade.

### EXAMPLE 4.9

The seed coat and part of the two cotyledons and primary leaves were removed from the seed.

### EXAMPLE 4.10

Part of two cotyledons and two primary leaves were removed, and the apical meristem was wounded using a scalpel blade.

### EXAMPLE 5

### Infection and Co-Cultivation of Soybean Seed Explants

The prepared explants from Example 4 were infected with Agrobacterium by mixing the explants with bacterial suspension as prepared in Example 3. The mixture was incubated for up to 24 hours at room temperature. Following infection, the explants were removed from the Agrobacterium suspension and placed on a co-cultivation medium, for example SoyCCM, SoyCoC. The co-cultivation plates were incubated for up to 7 days.

### EXAMPLE 6

### Regeneration and Selection of Transgenic Plants

After co-cultivation, elongated hypocotyls of the explants were trimmed back just below the cotyledon nodes. The explants were then transferred onto recovery medium with antibiotics to kill Agrobacterium or to inhibit Agrobacterium growth, without selection agent, such as SoyR1. The plates with the explants were incubated for up to 7 days at 24° C. under a 16/8 hour light/dark regimen, 80-160 μE/m²/s.

After the recovery period, elongated epicotyls or developing shoots were excised and transferred to regeneration media with selection agent, such as SoyR2, along with the cotyledon for 14 days. During the subculture step, one explant in recovery media can be separated into 2 or 3 pieces or kept in one piece and place into selection media SoyR2. SoyR2 media contained 6-8 mg/L glufosinate for selection.

After 2 weeks in regeneration/selection media such as SoyR2, developed or developing multiple shoot clusters were transferred to elongation medium, such as SoyE1 for shoot elongation. SoyE1 media contained 4-8 mg/L glufosinate. Subcultures to fresh elongation media were performed every two weeks.

Elongated shoots (>2 cm) were transferred to elongation media SoyE2 without selection for 14 days. After 14 days in SoyE2 media, shoots were transferred to rooting medium SoyRoot.

Leaves were sampled for Taqman analysis to identify transformants that contain the bar or pat gene. Taqman positive and rooted plants were rinsed with water to wash off the agar medium, and transplanted to soil and grown in the greenhouse for seeds.

### EXAMPLE 7

### Production of Transgenic Plants from Immature Seed Explants Without Primary Leaves

Transgenic plants were produced from isolated immature seed explants with primary leaves removed as described in Examples 4.3 or 4.4 using glufosinate selection in soybean variety Jack, Williams 82, and an elite variety S42H1, as set forth in Table 1. Transformants were identified by molecular analysis of leaf samples with Taqman analysis. Secondary Taqman analysis was further performed on multiple leaf samples of the greenhouse grown plants to confirm the presence of bar or pat gene (for 15237 and 15238) and the ZsGreen fluorescent protein gene for 15238.

TABLE 1
Transgenic Plants Produced from Immature Seed Explants

| | Experiment | | | No. | Events | Tranf. |
|---|---|---|---|---|---|---|
| Vector | ID | Genotype | Target | Explants | In GH | Effic. |
| 15237 | SYUK2006018571 | Williams 82 | Refrigerated | 62 | 1 | 1.6% |
| | | | immature seeds | | | |
| 15237 | SYUK2006018573 | Williams 82 | Refrigerated | 62 | 6 | 9.7% |
| | | | immature seeds | | | |
| 15237 | SYUK2006018525 | S42H1 | immature seeds | 57 | 3 | 5.3% |
| 15237 | SYUK2006018529 | S42H1 | immature seeds | 72 | 8 | 11.1% |
| 15237 | SYUK2006018531 | S42H1 | immature seeds | 75 | 6 | 8.0.% |
| 15238 | SYHT2006017193 | S42H1 | immature seeds | 50 | 2 | 4.0% |
| 15238 | SYHT2006017199 | S42H1 | immature seeds | 51 | 0 | 0% |
| 15238 | SYHT2006017685 | Jack | immature seeds | 124 | 3 | 2.4% |
| 15238 | SYHT2006017749 | Jack | immature seeds | 87 | 1 | 1.1% |
| 15238 | SYHT2006017751 | Jack | immature seeds | 45 | 5 | 11.1% |
| 15238 | SYHT2006017793 | Jack | immature seeds | 46 | 15 | 32.6% |
| 15238 | SYHT2006017795 | Jack | immature seeds | 40 | 1 | 2.5% |
| 15238 | SYHT2006017797 | Jack | immature seeds | 49 | 1 | 2.0% |

### EXAMPLE 8

### Production of Transgenic Plants from Split Half Seed Explants Without Primary Leaves

Transgenic plants were produced from immature split seed explants with primary leaves removed as described in Example 4.7. Transgenic plants were generated from soybean variety Jack and an elite variety S42H1, as set forth in Table 2.

TABLE 2
Transgenic Plants Produced from Split Half Seed Explants

| | | | | No. | Events in | Transf. |
|---|---|---|---|---|---|---|
| Vector | Experiment ID | Genotype | Target | Explants | GH | effic. |
| 15237 | SYUK2006018691 | Jack | Split immature | 353 | 7 | 2% |
| | | | seeds | | | |
| 15237 | SYUK2006018783 | Jack | Split immature | 155 | 2 | 1.3% |
| | | | seeds | | | |
| 15237 | SYUK2006018785 | Jack | Split immature | 132 | 2 | 1.5% |
| | | | seeds | | | |
| 15237 | SYUK2006018697 | S42H1 | Split immature | 174 | 6 | 3.4% |
| | | | seeds | | | |
| 15238 | SYUK2006018693 | Jack | Split immature | 90 | 1 | 1.1% |
| | | | seeds | | | |

### REFERENCES

The references listed below, as well as all references cited in the specification, are incorporated herein by reference in their entireties to the extent that they supplement, explain, provide a background for, or teach methodology, techniques, and/or compositions employed herein.

Alam & Cook (1990) Anal Biochem 188:245-254.

Ballas et al. (1989) Nucleic Acids Res. 17: 7891.

Brown et al. (1987) Cell 49: 603.

Chilton (1993) Proc. Natl. Acad. Sci. USA 90: 3119.

Cubitt et al. (1995) Trends Biochem Sci 20:448-455.

DeBlock et al. (1987) EMBO J. 6: 2513.

DeBlock et al. (1989) Plant Physiol. 91: 691.

Deuschle et al. (1989) Proc. Natl. Acad. Sci. USA 86: 5400.

Deuschle et al. (1990) Science 248: 480.

Figge et al. (1988) Cell 52: 713.

Fromm et al. (1990) BioTechnology 8: 833.

Fuerst et al. (1989) Proc. Natl. Acad. Sci. USA 86: 2549.

Gordon-Kamm et al. (1990) Plant Cell 2: 603.

Gruber et al., “Vectors for Plant Transformation” in Methods in Plant Molecular Biology and Biotechnology, Glick, B. R. and Thompson, J. E. Eds. (CRC Press, Inc., Boca Raton, 1993) pp. 89-119.

Guerineau et al. (1991) Mol. Gen. Genet. 262: 141.

Hooykaas (1989) Plant Mol. Biol. 13: 327.

Hu et al. (1987) Cell 48: 555.

Ishida et al. (1996) Nature Biotechnol. 14: 745.

Joshi et al. (1987) Nucleic Acids Res. 15: 9627.

Klein et al. (1987) Nature 327:70.

Komari et al. (1996) The Plant Journal 10:165.

O. Mayo, The Theory of Plant Breeding, Second Edition (Clarendon Press, Oxford, England (1987).

Miki et al., “Procedures for Introducing Foreign DNA into Plants” in Methods in Plant Molecular Biology and Biotechnology, Glick, B. R. and Thompson, J. E. Eds. (CRC Press, Inc., Boca Raton, 1993) pp. 67-88.

Mogen et al. (1990) Plant Cell 2:1261.

Mollony et al. (1993) Monograph Theor. Appl. Genet NY 19: 148.

Munroe et al. (1990) Gene 91: 151.

Murray et al. (1989) Nucleic Acids. Res. 17: 477.

Proudfoot (1991) Cell 64: 671.

Reznikoff (1992) Mol. Microbiol. 6: 2419.

Rose & Botstein (1983) Meth Enzymol 101:167-180.

Sandford et al. (1988) Particulate Science and Technology 5:27.

Sanfacon et al. (1991) Genes Dev. 5:141.

Smith et al. (1995) Crop Science 35: 301.

Welsh, J. R., Fundamentals of Plant Genetics and Breeding (John Wiley and Sons, New York, (1981)).

Wricke and Weber, Quantitative Genetics and Selection Plant Breeding (Walter de Gruyter and Co., Berlin (1986)).

Yao et al. (1992) Cell 71: 63.

Yarranton (1992) Curr. Opin. Biotech 3: 506 (1992).

U.S. Pat. No. 5,380,831.

U.S. Pat. No. 5,436,391.

U.S. Pat. No. 5,569,834.

U.S. Pat. No. 6,858,777.

PCT International Publication No. WO 97/47763.

European Patent Application No. EP 270,356.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. An organogenic method of transforming soybean cells or tissue, the method comprising: preparing an explant from an immature soybean seed, contacting the explant with a genetic construct, and culturing the explant in the presence of a selection agent.

2. The method of claim 1, wherein preparing the explant comprises one or more of the following:
a. isolating seeds from seed pods and sterilizing the seeds.
b. preparing transformation target explant tissues by one of the following,
i. removing from the seed its seed coat, one cotyledon, and two primary leaves;
ii. removing the hypocotyl from the seed, removing the seed coat, and removing one cotyledon and two primary leaves;
iii. removing the hypocotyl from the seed, removing part or all of the seed coat, bisecting the immature soybean seed longitudinally through an embryo axis, whereby both halves of the explant include one cotyledon and part of the immature embryo axis;
iv. removing the seed coat; and
v. removing the seed coat, and removing at least part of two cotyledons and two primary leaves.

3. The method of claim 2, wherein the one or more methods further comprise wounding a plumule, cotetylenary nodal region, hypocotyl, or combination thereof in the explant.

4. The method of claim 1, wherein the immature soybean seed is selected from developmental stage late R5, R6, or R7

5. The method of claim 1, wherein the genetic construct comprises a gene of interest and/or a selectable marker gene.

6. The method of claim 5, wherein the selectable marker gene confers antibiotic or herbicide resistance to the explant.

7. The method of claim 6, wherein the antibiotic is selected from the group consisting of: cefotaxime, timetin, vancomycin, carbenicillin, gentamicin, kanamycin, streptomycin, azithromycin, erythromycin, penicillin G, penicillin V, oxacillin, cloxacillin, dicloxacillin, ampicillin, amoxicillin, ticarcillin, ciprofloxacin, doxycycline, minocycline, tetracycline, vancomycin, and combinations thereof.

8. The method of claim 6, wherein the herbicide is selected from the group consisting of: glyphosate, sulfonylurea, imidazolinone, glufosinate, bialophos, phenoxy proprionic acid, cycloshexone, triazine, benzonitrile, HPPD inhibitors and combinations thereof.

9. The method of claim 1, wherein the genetic construct comprises a DNA molecule delivered by an Agrobacterium cell.

10. The method of claim 9, wherein the Agrobacterium is Agrobacterium tumefaciens.

11. The method of claim 9, wherein the explant is contacted with the Agrobacterium containing genetic vector for up to about 24 hours.

12. The method of claim 11, wherein the explants are further co-cultured with Agrobacterium for up to about 7 days.

13. The method of claim 1, wherein the contacting comprises delivering the genetic construct to the explant using a ballistic device.

14. The method of claim 1, further comprising culturing the explant on a culture medium comprising a plant hormone.

15. The method of claim 1, further comprising culturing the transformed shoot on a shooting medium comprising a plant hormone.

16. The method of claim 1, further comprising regenerating the shoot into a genetically transformed soybean plant.

17. A transgenic soybean cell or tissue prepared according to the method of claim 1.

18. A soybean plant regenerated from the transgenic soybean cell or tissue of claim 17.

19. A transgenic seed produced by the soybean plant of claim 18.
